(19) United States
(12) Patent Application Publication
(10) Pub. No.: US 2009/0306348 A1
(43) Pub. Date: Dec. 10, 2009

(54) Antibody Formulation

(75) Inventors: Joel Goldstein, Flemington, NJ (US); Arvind Srivastava, New York, NY (US)

Correspondence Address: ELI LILLY & COMPANY, PATENT DIVISION, P.O. BOX 6288, INDIANAPOLIS, IN, 46206-6288, US

(73) Assignee: ImClone Systems Incorporated, New York, NY (US)

(21) Appl. No.: 12/224,122

(22) PCT Filed: Feb. 15, 2007
(86) PCT No.: PCT/US07/04050

§ 371 Date: Mar. 27, 2009

Related U.S. Application Data
(60) Provisional application No. 60/774,101, filed on Feb. 15, 2006.

(51) Int. Cl. C07K 16/00

(52) U.S. Cl. 530/389.2

(57) ABSTRACT

The present invention provides formulations and methods for the stabilization of antibodies. In one embodiment, the invention provides the stabile formulation of antibodies that are prone to non-enzymatic fragmentation at the hinge region. In a further embodiment, the invention provides methods of stabilization of antibodies comprising lyophilizing an aqueous formulation of an antibody. The formulations can be lyophilized to stabilize the antibodies during processing and storage, and then the formulations can be reconstituted for pharmaceutical administration. In one embodiment, the present invention provides methods of stabilization of anti-VEGFR antibodies comprising lyophilizing an aqueous formulation of an anti-VEGFR antibody. The formulations can be lyophilized to stabilize the anti-VEGFR antibodies during processing and storage, and then the formulations can be reconstituted for pharmaceutical administration.

### CROSS REFERENCE TO RELATED APPLICATIONS

This application claims priority to U.S. Application No. 60/774,101, filed Feb. 15, 2006, which is incorporated herein by reference in its entirety.

### FIELD OF THE INVENTION

The present invention is directed to formulations and methods for the stabilization of antibodies. In one embodiment, the invention provides formulations and methods to stabilize antibodies that are prone to non-enzymatic cleavage at the hinge region. More particularly, this invention relates to the formulation of antibodies that are prone to non-enzymatic cleavage at the hinge region with a buffer and a lyoprotectant. In another embodiment, the invention provides methods and formulations for the stabilization of anti-VEGFR antibodies.

### BACKGROUND OF THE INVENTION

Antibodies in liquid formulations are susceptible to a variety of chemical and physical processes including hydrolysis, aggregation, oxidation, deamidation, and fragmentation at the hinge region. This fragmentation is a non-enzymatic process, which may be temperature and/or pH dependant, and typically occurs in the heavy chain hinge region near the papain cleavage site. These processes can alter or eliminate the clinical efficacy of therapeutic antibodies by decreasing the availability of functional antibodies, and by reducing or eliminating their antigen binding characteristics. The present invention addresses the need for stabile formulations of monoclonal antibodies, especially those that are prone to non-enzymatic cleavage at the hinge region, and further provides a method and formulation for lyophilizing these antibodies.

### BRIEF SUMMARY OF THE INVENTION

The present invention is directed to formulations and methods for the stabilization of antibody preparations. Further, the present invention is directed also to formulations and methods for the stabilization of antibodies that are prone to non-enzymatic cleavage, particularly at the hinge region.

In one embodiment, the invention provides a stabile formulation comprising an antibody that is prone to non-enzymatic cleavage, and a buffer. The formulation may also contain one or more stabilizing agents. In addition, the formulation may contain a surfactant.

In another embodiment, the invention provides a formulation that is compatible with lyophilization and may contain a lyoprotectant.

In another embodiment, the invention provides a lyophilized formulation comprising an antibody that is prone to non enzymatic cleavage, a histidine buffer, and a lyoprotecting sugar.

In another embodiment, the present invention provides methods of stabilization of antibodies that are prone to non-enzymatic cleavage, comprising formulation in a histidine buffer and a lyoprotecting sugar. In addition, the formulation may contain a surfactant. The formulations can be lyophilized to stabilize the antibodies during processing and storage, and then the formulations can be reconstituted for pharmaceutical administration. In a further embodiment, the reconstituted antibodies may be used in a multidose format.

In one embodiment, the invention provides a stabile lyophilized formulation comprising an anti-VEGFR antibody, a buffer, and a lyoprotectant. The formulation may also contain one or more stabilizing agents. In addition, the formulation may contain a surfactant.

In another embodiment, the invention provides a stabile lyophilized formulation comprising an anti-VEGFR2 antibody, a buffer, and a lyoprotectant. The formulation may also contain one or more stabilizing agents. In addition, the formulation may contain a surfactant.

In another embodiment, the invention provides a lyophilized formulation comprising an anti-VEGFR2 antibody, a histidine buffer, and a lyoprotecting sugar.

In another embodiment, the present invention provides methods of stabilization of anti-VEGFR antibodies comprising lyophilizing an aqueous formulation of an anti-VEGFR antibody. The formulations can be lyophilized to stabilize the anti-VEGFR antibodies during processing and storage, and then the formulations can be reconstituted for pharmaceutical administration

In another embodiment, the present invention provides a method of inhibiting VEGFR activity by providing a composition of the present invention. The present invention also provides a method of inhibiting the VEGF pathway in mammals, particularly humans, comprising administering a composition of the present invention. The present invention also provides a method of treating VEGFR-dependent conditions comprising administering a composition of the present invention.

### BRIEF DESCRIPTION OF THE FIGURES

FIG. 1 shows an size exclusion chromatography-high performance liquid chromatography (SEC-HPLC) chromatogram of the IMC-1121B antibody in PBS after 3 months of incubation at 40° C.

FIG. 2 shows the amino acid sequence for the heavy chain of IMC-1121B, and the sites where non-enzymatic cleavage occurs.

FIG. 3 shows SDS-PAGE of degraded IMC-1121B and its size exclusion fractions.

FIG. 4 shows a regression plot for DSC analysis of IMC-1121B.

FIG. 5 shows a prediction profiler for an agitation study.

FIG. 6 shows a prediction profiler of real-time, accelerated temperature stability of IMC-1121B at 40° C.

FIG. 7 shows a prediction profiler of real-time accelerated temperature stability of IMC-1121B at −20° C.

FIG. 8 is an SEC-HPLC chromatogram of IMC-1121B following incubation for 150 days at 40° C. and at room temperature in PBS and 10 mM histidine buffer (pH 6.0).

FIG. 9 shows the variation of percent monomer of IMC-1121B in PBS and 10 mM histidine buffer (pH 6.0) as a function of incubation time at 40° C. and room temperature.

FIG. 10 shows the variation of percent aggregate of IMC-1121B in PBS and 10 mM histidine buffer (pH 6.0) as a function of incubation time at 40° C. and room temperature.

FIG. 11 shows the variation of percent degradent of IMC-1121B in PBS and 10 mM histidine buffer (pH 6.0) as a function of incubation time at 40° C. and room temperature.

FIG. 12 is an IEC-HPLC chromatogram of IMC-1121B after 30 and 150 days of incubation at RT and 40° C.

FIG. 13 shows reducing and non-reducing SDS-PAGE of IMC-1121B in PBS and 10 mM histidine buffer (pH 6.0) after 150 days of incubation at RT and 40° C.

FIG. 14 shows isoelectric focusing of IMC-1121B after 150 days of incubation at RT and 40° C.

FIG. 15 shows the freeze-drying cycle for IMC-1121B lyophilization process

FIG. 16 shows the percent monomer remaining for IMC-1121B lyophilized products after 100 days of incubation at 40° C. and 50° C.

FIG. 17 shows the percent monomer remaining for IMC-1121B in lyophilized and solution formulations as a function of incubation time at 50° C.

FIG. 18 shows the percent aggregates for IMC-1121B in lyophilized and solution formulations as a function of incubation time at 50° C.

FIG. 19 shows the percent degradents for IMC-1121B in lyophilized and solution formulations as a function of incubation time at 50° C.

FIG. 20 shows the percent monomer remaining for IMC-1121B in lyophilized and solution formulations as a function of incubation time at 40° C.

FIG. 21 shows the percent aggregates for INC-1121B in lyophilized and solution formulations as a function of incubation time at 40° C.

FIG. 22 shows the percent degradents for IMC-1121B in lyophilized and solution formulations as a function of incubation time at 40° C.

FIG. 23 shows the percent monomer remained for lyophilized and solution formulated IMC-1121B after incubation at 50° C.

FIG. 24 shows the percent aggregate for lyophilized and solution formulated IMC-1121B after incubation at 50° C.

FIG. 25 shows the percent degradent for lyophilized and solution formulated IMC-1121B after incubation at 50° C.

FIG. 26 shows the percent monomer remaining for lyophilized and solution formulated IMC-1121B after incubation at 40° C.

FIG. 27 shows the percent aggregate for IMC-1121B incubated at 40° C. in solution and freeze-dried formulations.

FIG. 28 shows the percent degradent for lyophilized and solution formulated IMC-1121B after incubation at 40° C.

FIG. 29 is an IEC-HPLC chromatogram of IMC-1121B incubated for 3 months at 40° C. in solution and freeze-dried formulations.

FIG. 30 shows the percent monomer remaining for lyophilized and solution formulated IMC-1121B after room temperature incubation.

FIG. 31 shows the percent aggregates for lyophilized and solution formulated IMC-1121B after room temperature incubation.

FIG. 32 shows the percent degradents for lyophilized and solution formulated 1121B after room temperature incubation.

FIG. 33 is an IEC-HPLC chromatogram of IMC-1121B in solution and freeze-dried formulations after incubation at room temperature for 3 months.

FIG. 34 shows reducing SDS-PAGE of IMC-1121B in solution and freeze-dried formulations after incubation at room temperature, 40° C. and 50° C. for 3 months.

FIG. 35 shows non-reducing SDS-PAGE of IMC-1121B in solution and freeze-dried formulations after incubation at room temperature, 40° C. and 50° C. for 3 months.

### DETAILED DESCRIPTION OF THE INVENTION

The present invention provides formulations for the freeze-drying of antibodies, including functional fragments thereof, that are prone to non-enzymatic cleavage. The formulations may comprise additional elements such as stabilizing agents, surfactants, reducing agents, carriers, preservatives, amino acids, and chelating agents. The present invention also provides methods of stabilizing an antibody composition comprising lyophilizing an aqueous formulation of an antibody in the presence of a lyoprotectant. The formulations may be lyophilized to stabilize the antibodies during processing and storage, and then reconstituted prior to pharmaceutical administration. Preferably, the antibody substantially retains its physical and chemical stability and integrity from production to administration. Various formulation components may be suitable to enhance stability according to the present invention, including buffers, surfactants, sugars, sugar alcohols, sugar derivatives, and amino acids. Various formulation properties may be suitable to enhance stability according to the present invention, including pH and concentration of formulation components.

According to the present invention, a buffer may be used to maintain the pH of the formulation. The buffer minimizes fluctuations in pH due to external variations. The formulations of the present invention contain one or more buffers to provide the formulations at a suitable pH, preferably about 5.5 to about 6.5, and most preferably about 6.0. Exemplary buffers include, but are not limited to organic buffers generally, such as histidine, citrate, malate, tartrate, succinate, and acetate. In one embodiment the buffer concentration is about 5 mM to about 50 mM. In a further embodiment the buffer concentration is about 10 mM.

The formulations of the present invention may contain one or more stabilizing agents, which may help prevent aggregation and degradation of the antibodies. Suitable stabilizing agents include, but are not limited to polyhydric sugars, sugar alcohols, sugar derivatives, and amino acids. Preferred stabilizing agents include, but are not limited to aspartic acid, lactobionic acid, glycine, trehalose, mannitol, and sucrose.

The formulations of the present invention may contain one or more surfactants. Antibody solutions have high surface tension at the air-water interface. In order to reduce this surface tension, antibodies tend to aggregate at the air-water interface. A surfactant minimizes antibody aggregation at the air-water interface, thereby helping to maintain the biological activity of the antibody in solution. For example, adding 0.01%. Tween 80 can reduce antibody aggregation in solution. When the formulation is lyophilized, the surfactant may also reduce the formation of particulates in the reconstituted formulation. In the lyophilized formulations of the present invention, the surfactant can be added to one or more of the pre-lyophilized formulation, the lyophilized formulation, and the reconstituted formulation, but preferably the pre-lyophilized formulation. For example, 0.005% Tween 80 can be added to the antibody solution before lyophilization. Surfactants include, but are not limited to Tween 20, Tween 80, Pluronic. F-68, and bile salts. In one embodiment, the surfactant concentration is about 0.001% to about 1.0%.

The lyophilization process can generate a variety of stresses that may denature proteins or polypeptides. These stresses include temperature decrease, ice crystal formation, ionic strength increase, pH changes, phase separation, removal of hydration shell, and concentration changes. Antibodies that are sensitive to the stresses of the freezing and/or drying process can be stabilized by adding one or more lyoprotectants. A lyoprotectant is a compound that protects against the stresses associated with lyophilization. Therefore lyoprotectants as a class include cryoprotectants, which just protect from the freezing process. One or more lyoprotectants may be used to protect from the stresses associated with lyophilization and may be, for example, a sugar such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and manmitol; propylene glycol; polyethylene glycol; Pluronics; and combinations thereof. Examples of preferred lyoprotectants include, but are not limited to the stabilizing agents and surfactants as described above.

The present invention provides stabilized formulations, which may be prepared through the process of lyophilization. Lyophilization is a stabilizing process in which a substance is first frozen and then the quantity of the solvent is reduced, first by sublimation (the primary drying process) and then desorption (the secondary drying process) to values that will no longer support biological activity or chemical reactions. In a lyophilized formulation, the hydrolysis, deamidation, oxidation and fragmentation reactions associated with solutions can be avoided or slowed significantly. A lyophilized formulation may also avoid damage due to short-term temperature fluctuations during shipping and allow for room temperature storage. The formulations of the present invention may also be dried by other methods known in the art such as spray drying and bubble drying. Unless otherwise specified, the formulations of the present invention are described in terms of their component concentrations as measured in the formulation before lyophilization.

In one embodiment, the present invention provides for methods and formulations to stabilize antibodies that are prone to non-enzymatic degradation, which may occur at the hinge region. Factors that may predispose an antibody to non-enzymatic cleavage include amino acid sequence, conformation and post-translational processing. Determination that an antibody undergoes non-enzymatic cleavage may be accomplished by incubation of the antibody in an aqueous solution. Typically, the incubation is performed at elevated temperatures to shorten the duration of the study. For example, incubation for 3 months at 40° C. or 50° C. Following the incubation, the degradation products may be analyzed using size exclusion chromatography-high performance liquid chromatography (SEC-HPLC).

Various analytical techniques known in the art can measure the antibody stability of a reconstituted lyophilized formulation. Such techniques include, for example, determining (i) thermal stability using differential scanning calorimetry (DSC) to determine the main melting temperature (Tm); (ii) mechanical stability using controlled agitation at room temperature; (iii) real-time isothermal accelerated temperature stability at temperatures of about −20° C., about 4° C., room temperature (about 23° C.-27° C.), about 40° C., and about 50° C.; (iv) solution turbidities by monitoring absorbance at about 350 nm and (v) the amount of monomer, aggregates and degradants using SEC-HPLC. Stability can be measured at a selected temperature for a selected time period.

In one embodiment, the lyophilized formulation provides a high concentration of the antibody upon reconstitution. In a further embodiment, the stable lyophilized formulation is reconstitutable with a liquid to form a solution with an antibody concentration about 1-10 times higher than the antibody concentration of the formulation before lyophilization. For instance, in one embodiment, the lyophilized formulation is reconstituted with 1 mL of water or less to obtain a particle-free reconstituted formulation with an antibody concentration of about 50 mg/mL to about 200 mg/mL.

Naturally occurring antibodies typically have two identical heavy chains and two identical light chains, with each light chain covalently linked to a heavy chain by an interchain disulfide bond. Multiple disulfide bonds further link the two heavy chains to one another. Individual chains can fold into domains having similar sizes (110-125 amino acids) and structures, but different functions. The light chain can comprise one variable domain (V_{L}) and/or one constant domain (C_{L}). The heavy chain can also comprise one variable domain (V_{H}) and/or, depending on the class or isotype of antibody, three or four constant domains (C_{H}1, C_{H} 2, C_{H}3 and C_{H}4). In humans, the isotypes are IgA, IgD, IgE, IgG, and IgM, with IgA and IgG further subdivided into subclasses or subtypes (IgA₁₋₂ and IgG₁₋₄).

Generally, the variable domains show considerable amino acid sequence variability from one antibody to the next, particularly at the location of the antigen-binding site. Three regions, called hypervariable or complementarity-determining regions (CDRs), are found in each of V_{L} and V_{H}, which are supported by less variable regions called framework variable regions.

The portion of an antibody consisting of V_{L} and V_{H} domains is designated Fv (fragment variable) and constitutes the antigen-binding site. Single chain Fv (scFv) is an antibody fragment containing a V_{L} domain and a V_{H} domain on one polypeptide chain, wherein the N terminus of one domain and the C terminus of the other domain are joined by a flexible linker (see, e.g., U.S. Pat. No. 4,946,778 (Ladner et al.); WO 88/09344, (Huston et al.). WO 92/01047 (McCafferty et al.) describes the display of scFv fragments on the surface of soluble recombinant genetic display packages, such as bacteriophage.

Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Multiple single chain antibodies, each single chain having one V_{H} and one V_{L} domain covalently linked by a first peptide linker, can be covalently linked by at least one or more peptide linker to form a multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of amino acid residues is about one hundred.

Two single chain antibodies can be combined to form a diabody, also known as a bivalent dimer. Diabodies have two chains and two binding sites, and can be monospecific or bispecific. Each chain of the diabody includes a V_{H} domain connected to a V_{L} domain. The domains are connected with linkers that are short enough to prevent pairing between domains on the same chain, thus driving the pairing between complementary domains on different chains to recreate the two antigen-binding sites.

Three single chain antibodies can be combined to form triabodies, also known as trivalent trimers. Triabodies are constructed with the amino acid terminus of a V_{L} or V_{H} domain directly fused to the carboxyl terminus of a V_{L} or V_{H} domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific.

Fab (Fragment, antigen binding) refers to the fragments of the antibody consisting of V_{L} C_{L} V_{H} and C_{H}1 domains. Those generated following papain digestion simply are referred to as Fab and do not retain the heavy chain hinge region. Following pepsin digestion, various Fabs retaining the heavy chain hinge are generated. Those divalent fragments with the interchain disulfide bonds intact are referred to as F(ab′)₂, while a monovalent Fab′ results when the disulfide bonds are not retained. F(ab′)₂ fragments have higher avidity for antigen that the monovalent Fab fragments.

Fc (Fragment crystallization) is the designation for the portion or fragment of an antibody that comprises paired heavy chain constant domains. In an IgG antibody, for example, the Fc comprises C_{H}2 and C_{H}3 domains. The Fc of an IgA or an IgM antibody further comprises a C_{H4} domain. The Fc is associated with Fc receptor binding, activation of complement-mediated cytotoxicity, and antibody-dependent cellular-cytoxicity (ADCC). For antibodies such as IgA and IgM, which are complexes of multiple IgG like proteins, complex formation requires Fc constant domains.

Finally, the hinge region separates the Fab and Fc portions of the antibody, providing for mobility of Fabs relative to each other and relative to Fc, as well as including multiple disulfide bonds for covalent linkage of the two heavy chains.

Thus, antibodies of the invention include, but are not limited to, naturally occurring antibodies, bivalent fragments such as (Fab′)₂, monovalent fragments such as Fab, single chain antibodies, single chain Fv (scFv), single domain antibodies, multivalent single chain antibodies, diabodies, triabodies, and the like that bind specifically with antigens.

Antibodies, or fragments thereof, of the present invention, for example, can be monospecific or bispecific. Bispecific antibodies (BsAbs) are antibodies that have two different antigen-binding specificities or sites. Where an antibody has more than one specificity, the recognized epitopes can be associated with a single antigen or with more than one antigen. Thus, the present invention provides bispecific antibodies, or fragments thereof, that bind to two different antigens

Specificity of antibodies, or fragments thereof, can be determined based on affinity and/or avidity. Affinity, represented by the equilibrium constant for the dissociation of an antigen with an antibody (K_{d}), measures the binding strength between an antigenic determinant and an antibody-binding site. Avidity is the measure of the strength of binding between an antibody with its antigen. Avidity is related to both the affinity between an epitope with its antigen binding site on the antibody, and the valence of the antibody, which refers to the number of antigen binding sites of a particular epitope. Antibodies typically bind with a dissociation constant (K_{d}) of 10⁻⁵ to 10⁻¹¹ liters/mol. Any K_{d} less than 10⁻⁴ liters/mol is generally considered to indicate nonspecific binding. The lesser the value of the K_{d}, the stronger the binding strength between an antigenic determinant and the antibody binding site.

As used herein, “antibodies” and “antibody fragments” includes modifications that retain specificity for a specific antigen. Such modifications include, but are not limited to, conjugation to an effector molecule such as a chemotherapeutic agent (e.g., cisplatin, taxol, doxorubicin) or cytotoxin (e.g., a protein, or a non-protein organic chemotherapeutic agent). The antibodies can be modified by conjugation to detectable reporter moieties. Also included are antibodies with alterations that affect non-binding characteristics such as half-life (e.g., pegylation).

Proteins and non-protein agents may be conjugated to the antibodies by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990) for the conjugation of doxorubicin and those described by Arnon et al., Adv. Exp. Med. Biol. 303, 79-90 (1991) and by Kiseleva et al., Mol. Biol. (USSR) 25, 508-514 (1991) for the conjugation of platinum compounds.

Antibodies of the present invention further include those for which binding characteristics have been improved by direct mutation, methods of affinity maturation, phage display, or chain shuffling. Affinity and specificity can be modified or improved by mutating CDRs and screening for antigen binding sites having the desired characteristics (see, e.g., Yang et al., J. Mol. Biol., 254: 392-403 (1995)). CDRs are mutated in a variety of ways. One way is to randomize individual residues or combinations of residues so that in a population of otherwise identical antigen binding sites, all twenty amino acids are found at particular positions. Alternatively, mutations are induced over a range of CDR residues by error prone PCR methods (see, e.g., Hawkins et al., J. Mol. Biol., 226: 889-896 (1992)). For example, phage display vectors containing heavy and light chain variable region genes can be propagated in mutator strains of E. coli (see, e.g., Low et al., J. Mol. Biol., 250: 359-368 (1996)). These methods of mutagenesis are illustrative of the many methods known to one of skill in the art.

Each domain of the antibodies of this invention can be a complete immunoglobulin domain (e.g., a heavy or light chain variable or constant domain), or it can be a functional equivalent or a mutant or derivative of a naturally-occurring domain, or a synthetic domain constructed, for example, in vitro using a technique such as one described in WO 93/11236 (Griffiths et al.). For instance, it is possible to join together domains corresponding to antibody variable domains, which are missing at least one amino acid. The important characterizing feature of the antibodies is the presence of an antigen binding site. The terms variable heavy and light chain fragment should not be construed to exclude variants that do not have a material effect on specificity.

Antibodies and antibody fragments of the present invention can be obtained, for example, from naturally occurring antibodies, or Fab or scFv phage display libraries. It is understood that, to make a single domain antibody from an antibody comprising a V_{H} and a V_{L} domain, certain amino acid substitutions outside the CDRs can be desired to enhance binding, expression or solubility. For example, it can be desirable to modify amino acid residues that would otherwise be buried in the V_{H}-V_{L} interface.

Further, antibodies and antibody fragments of the invention can be obtained by standard hybridoma technology (Harlow & Lane, ed., Antibodies: A Laboratory Manual, Cold Spring Harbor, 211-213 (1998), which is incorporated by reference herein) using transgenic mice (e.g., KM mice from Medarex, San Jose, Calif.) that produce human immunoglobulin gamma heavy and kappa light chains. In a preferred embodiment, a substantial portion of the human antibody producing genome is inserted into the genome of the mouse, and is rendered deficient in the production of endogenous murine antibodies. Such mice may be immunized subcutaneously (s.c.) with part or all of target molecule in complete Freund's adjuvant.

The present invention also provides a method of treatment comprising administering a reconstituted formulation. The reconstituted formulations are prepared by reconstituting the lyophilized formulations of the present invention, for example with 1 mL water. The reconstitution time is preferably less than 1 minute. The concentrated reconstituted formulation allows for flexibility in administration. For example, the reconstituted formulation can be administered in a dilute form intravenously, or it can be administered in a more concentrated form by injection. A concentrated reconstituted formulation of the present invention can be diluted to a concentration that is tailored to the particular subject and/or the particular route of administration. Accordingly, the present invention provides methods of treatment comprising administering a therapeutically effective amount of an antibody to a mammal, particularly a human, in need thereof. The term administering as used herein means delivering the antibody composition of the present invention to a mammal by any method that can achieve the result sought. The reconstituted formulation can be administered, for example, intravenously or intramuscularly. In one embodiment, a concentrated reconstituted formulation is administered by injection.

Antibodies of the present invention are preferably human. In one embodiment the composition of the present invention may be used to treat neoplastic diseases, including solid and non-solid tumors and for treatment of hyperproliferative disorders.

Therapeutically effective amount means an amount of antibody of the present invention that, when administered to a mammal, is effective in producing the desired therapeutic effect, such as reducing or neutralizing VEGFR activity, inhibition of tumor growth, or treating a non-cancerous hyperproliferative disease. Administration of the antibodies as described above can be combined with administration of other antibodies or any conventional treatment agent, such as an anti-neoplastic agent.

In an embodiment of the invention, the composition can be administered in combination with one or more anti-neoplastic agents. Any suitable anti-neoplastic agent can be used, such as a chemotherapeutic agent, radiation or combinations thereof. The anti-neoplastic agent can be an alkylating agent or an anti-metabolite. Examples of alkylating agents include, but are not limited to, cisplatin, cyclophosphamide, melphalan, and dacarbazine. Examples of anti-metabolites include, but not limited to, doxorubicin, daunorubicin, paclitaxel, irinotecan (CPT-11), and topotecan. When the anti-neoplastic agent is radiation, the source of the radiation can be either external (external beam radiation therapy—EBRT) or internal (brachytherapy—BT) to the patient being treated. The dose of anti-neoplastic agent administered depends on numerous factors, including, for example, the type of agent, the type and severity tumor being treated and the route of administration of the agent. It should be emphasized, however, that the present invention is not limited to any particular dose.

Antibodies of the present invention may be, but are not limited to, antibodies to VEGFR, IGF-IR, EGFR and PDGFR.

In one embodiment, the antibodies, or fragments thereof, of the present invention are specific for VEGFR. In another embodiment, the present invention provides bispecific antibodies, or fragments thereof, that bind to two different antigens, with at least one specificity for VEGFR. VEGFR refers to the family of human VEGF receptors, including VEGFR-1 (FLT1), VEGFR-2 (KDR), VEGFR-3 (FLT4).

Vascular endothelial growth factor (VEGF) is a key mediator of angiogenesis. In healthy humans, VEGF promotes angiogenesis in the developing embryo, in healing wounds, and during female reproductive cycling. However, VEGF mediates angiogenesis in tumors when it is upregulated by oncogene expression, growth factors, and hypoxia. Angiogenesis is essential for tumor growth past a certain size by the limited diffusion of nutrients and oxygen.

Thus, in one embodiment, the anti-VEGFR antibody binds VEGFR and blocks binding of a ligand, such as VEGF. This blockage may result in inhibition of tumor growth, which includes inhibition of tumor invasion, metastasis, cell repair, and angiogenesis, by interfering with the effects of VEGFR activation.

In one embodiment, the antibody is the anti-VEGFR-2 (KDR) antibody, IMC-1121B (IgG1), which is disclosed in WO 03/07840 (PCT/US03/06459). The nucleotide and amino acid sequence of the V_{H} for IMC-1121B are represented in SEQ ID NOS 1 and 2, respectively. The nucleotide and amino acid sequence of the V_{L} for IMC-1121B are represented in SEQ ID NOS 3 and 4, respectively.

Equivalents of the antibodies, or fragments thereof, of the present invention also include polypeptides with amino acid sequences substantially the same as the amino acid sequence of the variable or hypervariable regions of the full-length anti-VEGFR antibody provided herein. Substantially the same amino acid sequence is defined herein as a sequence with at least about 70%, preferably at least about 80%, and more preferably at least about 90% homology, as determined by the FASTA search method in accordance with Pearson and Lipman (Proc. Natl. Acad. Sci. USA 85, 2444-8 (1988)).

### EXAMPLES

The following examples further illustrate the invention, but should not be construed to limit the scope of the invention in any way. Detailed descriptions of conventional methods, such as those employed in the analysis of proteins can be obtained from numerous publications such as Current Protocols in Immunology (published by published by John Wiley & Sons). All references mentioned herein are incorporated in their entirety.

### Example 1

### Fragmentation of Anti-VEGFR-2 Antibody, IMC-1121B

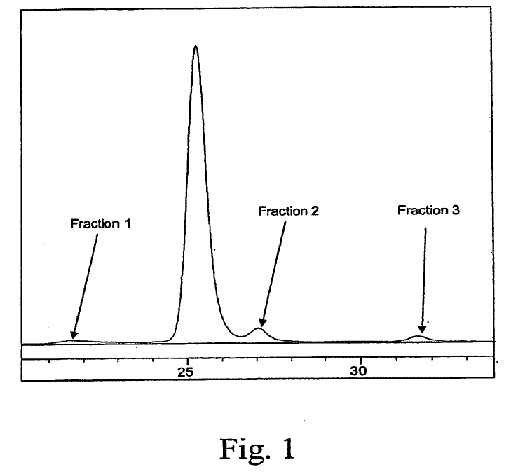

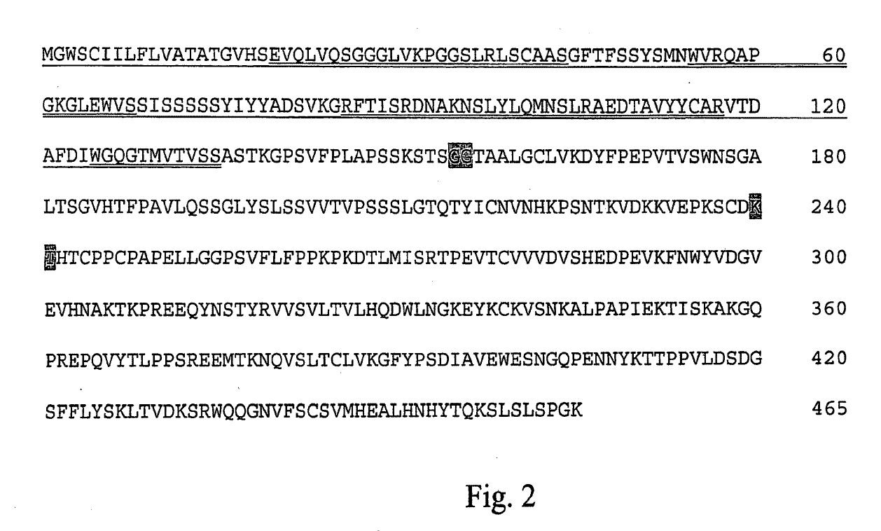

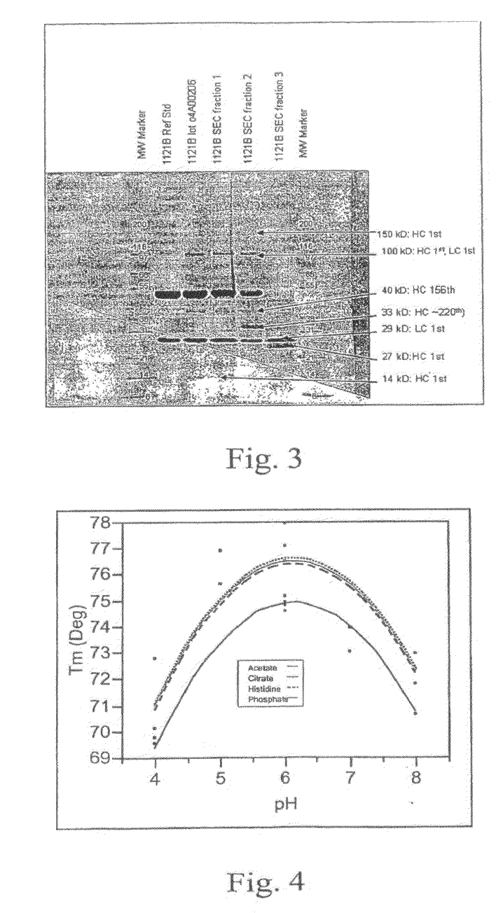

IMC-1211B at 5 mg/mL in phosphate-buffered saline (PBS) was incubated at 40° C. for 3 months. Following this incubation, SEC-HPLC and N-terminal sequencing were used to analyze the degradation products. The SEC-HPLC chromatogram of degraded IMC-1121B in PBS is shown in FIG. 1. The degraded product has two degradent peaks (fractions 2 and 3) in addition to aggregate (fraction 1) and monomer peaks. The fractions were collected using a fraction collector for N-terminal sequence analysis. The cDNA sequence for IMC-1121B heavy chain is shown in FIG. 2. Signal sequence, variable regions and constant regions are shown with underlined, double-underlined and plain text, respectively. N-terminal sequencing analysis of the degraded sample and fractions 2 and 3 has shown two sites of fragmentation in the heavy chain (grey-highlighted text in FIG. 2). The site at the 156^{th} residue from the N-terminus results in two heavy chain fragments detected on reduced SDS-PAGE (FIG. 3) as about 40 KD and about 15 KD bands. The other fragmentation site in the hinge region at the 220^{th} residue from the N-terminus results in about 33 KD and about 27 KD bands on reduced SDS-PAGE (FIG. 3).

### Example 2

### Optimization of Buffer Formulation

The freeze-dried formulation for IMC-1121B was developed in two stages. In the first stage, the solvent buffer was optimized using a design of experiment approach (DOE) with fractional factorial modeling as outlined in Table 1. The factors screened in this optimization process were buffer, pH, salt, amino acids, surfactants sugars, and sugar derivatives. Solvent optimization was performed at a 1121B concentration of 5 mg/mL. Controlled agitation at 300 rpm at room temperature was used to test mechanical stability. Thermal stability was tested using DSC and accelerated temperatures. The DOE predictions were confirmed using traditional one-factor-at-a-time methodology. Linear regression analysis was used to determine the significance of the results.

TABLE 1
Design of experiment (DOE) matrix

| | | NaCl | Aspartic | Lactobionic | Tween | Glycine | Arginine | Mannitol | Sucrose | Trehalose |
|---|---|---|---|---|---|---|---|---|---|---|
| Buffer type | pH | (mM) | acid (%) | acid (%) | 80 (%) | (%) | (%) | (%) | (%) | (%) |
| Citrate | 6 | 150 | 0 | 0 | 0.5 | 2 | 2 | 2 | 0 | 0 |
| Citrate | 4 | 0 | 0.5 | 0 | 0 | 2 | 2 | 0 | 0 | 2 |
| Citrate | 4 | 0 | 0.5 | 0.5 | 0.5 | 0 | 0 | 2 | 2 | 0 |
| Citrate | 6 | 150 | 0 | 0.5 | 0 | 0 | 0 | 0 | 2 | 2 |
| Citrate | 5 | 75 | 0.25 | 0.25 | 0.25 | 1 | 1 | 1 | 1 | 1 |
| Acetate | 6 | 0 | 0 | 0.5 | 0.5 | 2 | 0 | 0 | 0 | 0 |
| Acetate | 5 | 75 | 0.25 | 0.25 | 0.25 | 1 | 1 | 1 | 1 | 1 |
| Acetate | 4 | 150 | 0.5 | 0.5 | 0 | 2 | 0 | 2 | 0 | 2 |
| Acetate | 6 | 0 | 0 | 0 | 0 | 0 | 2 | 2 | 2 | 2 |
| Acetate | 4 | 150 | 0.5 | 0 | 0.5 | 0 | 2 | 0 | 2 | 0 |
| Histidine | 7 | 75 | 0.25 | 0.25 | 0.25 | 1 | 1 | 1 | 1 | 1 |
| Histidine | 8 | 0 | 0.5 | 0 | 0.5 | 2 | 0 | 0 | 2 | 2 |
| Histidine | 5 | 150 | 0 | 0.5 | 0.5 | 0 | 2 | 0 | 0 | 2 |
| Histidine | 6 | 150 | 0 | 0 | 0 | 2 | 0 | 2 | 2 | 0 |
| Histidine | 8 | 0 | 0.5 | 0.5 | 0 | 0 | 2 | 2 | 0 | 0 |
| Phosphate | 7 | 75 | 0.25 | 0.25 | 0.25 | 1 | 1 | 1 | 1 | 1 |
| Phosphate | 8 | 150 | 0.5 | 0.5 | 0.5 | 2 | 2 | 2 | 2 | 2 |
| Phosphate | 6 | 0 | 0 | 0.5 | 0 | 2 | 2 | 0 | 2 | 0 |
| Phosphate | 6 | 0 | 0 | 0 | 0.5 | 0 | 0 | 2 | 0 | 2 |
| Phosphate | 8 | 150 | 0.5 | 0 | 0 | 0 | 0 | 0 | 0 | 0 |
| PBS | 7.2 | 145 | 0 | 0 | 0 | 0 | 0 | 0 | 0 | 0 |

Differential scanning calorimetry (DSC) study: The melting, or transition, temperature (Tm) was measured using a MicroCal VP-DSC. The protein concentration was set at 5 mg/mL and temperature ramping was from 5° C. to 95° C. at a scan rate of 1.5° C./min. The thermal melting curves of IMC-1121B in various formulations (Table 1) were collected. The melting temperatures corresponding to the main transition peak (50% of the molecules are denatured) were fitted to a linear regression model to estimate the effect of tested variables on Tm. The model was statistically significant with a p=0.0006. The significant factors (p<0.05) were pH and buffer type. The regression plot for the Tm variation with buffer type and pH is shown in FIG. 4. The optimal pH was approximately 6.0 for the histidine, citrate and acetate buffers, which were superior to phosphate buffer at pH 6.0. Other variables did not have statistically significant effect on Tm.

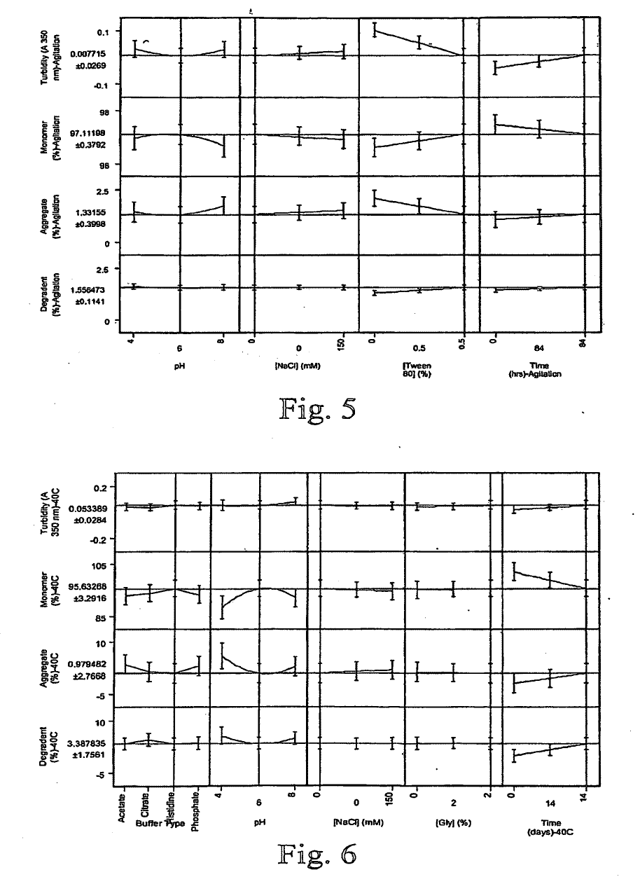

Agitation study: Antibody solutions were agitated on a platform shaker at 300 rpm at room temperature. Five mL of IMC-1121B at 5 mg/mL in a 20 mL glass vial was agitated in various formulations (Table 1) for up to 84 hours. Solution turbidity, percent monomer, percent aggregate, and percent degradent were determined as follows. Solutions turbidity was measured by absorbance at 350 nm using Shimatzu 1601 biospec spectrophotometer. Percent monomer, percent aggregate, and percent degradent were measured using SEC-HPLC performed on an Agilent 1100 Series LC using Tosoph Biosep TSK 3000 column with 10 mM sodium phosphate, 0.5M CsCl, at pH 7.0 as the mobile phase. The effect of tested variables on turbidity, percent monomer, aggregate and degradent were estimated by fitting to a linear regression model using JMP software (SAS institute, NC). The p-value for the Actual by Predicted plot was <0.002. The effects of the significant variables pH, Tween 80, NaCl and time on turbidity, percent monomer, aggregate and degradent are shown in FIG. 5.

Real-time, accelerated temperature stability at 40° C.: The IMC-1121B at 5 mg/mL in various formulations (Table 1) were incubated at 40° C. for up to 14 days. The solution turbidity, percent monomer, aggregate and degradent were determined as described above. The effect of tested variables on turbidity, percent monomer, aggregate and degradent were estimated by fitting it to a linear regression model using JMP software. The p value for Actual by Predicted plots were <0.001. The effect of significant variables on turbidity, percent monomer, aggregate and degradent are shown in FIG. 6. The optimal buffer is histidine at pH 6.0. Salt reduced monomer and increased aggregation. But did not affect degradation. Glycine has no effect on monomer, aggregate or degradent.

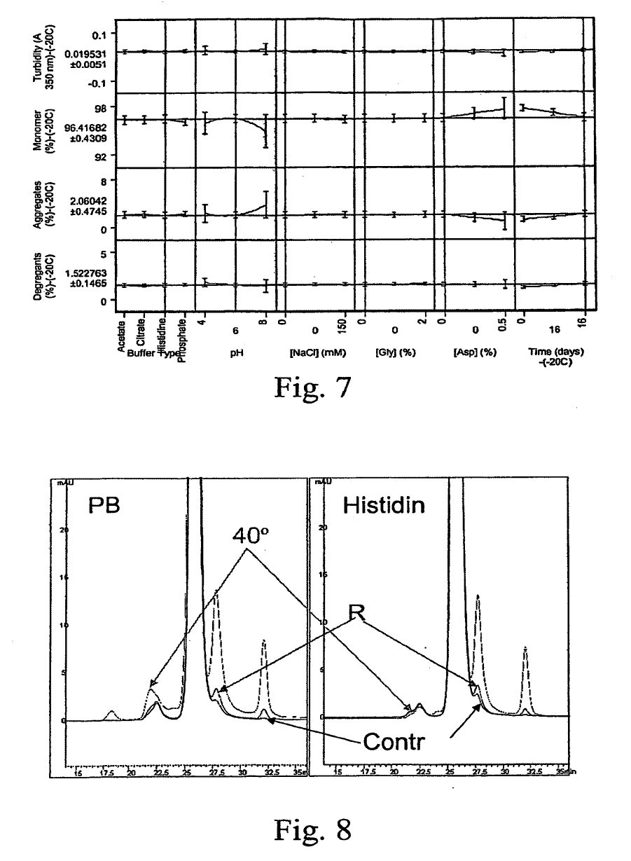

Real-time freezing temperature stability at −20° C.: The IMC-1121B antibody at 5 mg/mL in various formulations (Table 1) were incubated at −20° C. for up to 16 days. The solution turbidity, percent monomer, aggregate and degradent was estimated as described above. The effect of tested variables on turbidity, percent monomer, aggregate and degradent were determined by fitting to a linear regression model using JMP software. The p-value for Actual by Predicted plot was <0.001. The effect of significant variables on turbidity, percent monomer, aggregate and degradent are shown in FIG. 7. The optimal pH was 6.0. Aspartic acid increased monomer and decreased aggregation with a negligible effect on degradation. NaCl and glycine had negligible effect on turbidity, monomer, aggregate and degradent.

### Example 3

### Comparison of IMC-1121B Stability in PBS and 10 mM Histidine Buffer (pH 6.0) Formulations

DOE screening studies predicted that the IMC-1121B antibody has significantly better stability in a 101 mM histidine buffer (pH 6.0) formulation than in PBS. In this study, the stability of IMC-1121B at 5 mg/mL concentration in 10 mM histidine pH 6.0 and PBS was examined by various techniques to confirm the DOE prediction.

Differential scanning calorimetry (DSC) study: Thermal stability of IMC-1121B in PBS and 10 mM histidine buffer (pH 6.0) formulations were examined according to the procedure described in Methods. The melting temperatures for main transition were 70.0 and 76.6° C. for IMC-1121B in PBS and 10 mM histidine buffer (pH 6.0), respectively.

Real-time accelerated temperature stability at 40° C. and room temperature: The IMC-1121B at 5 mg/mL was incubated at 40° C. and room temperature (RT) for up to 150 days in PBS and 10 mM histidine buffer (pH 6.0) formulations. Following incubation, the samples were analyzed by SEC-HPLC, IEC-HPLC, SDS-PAGE and IEF as described below.

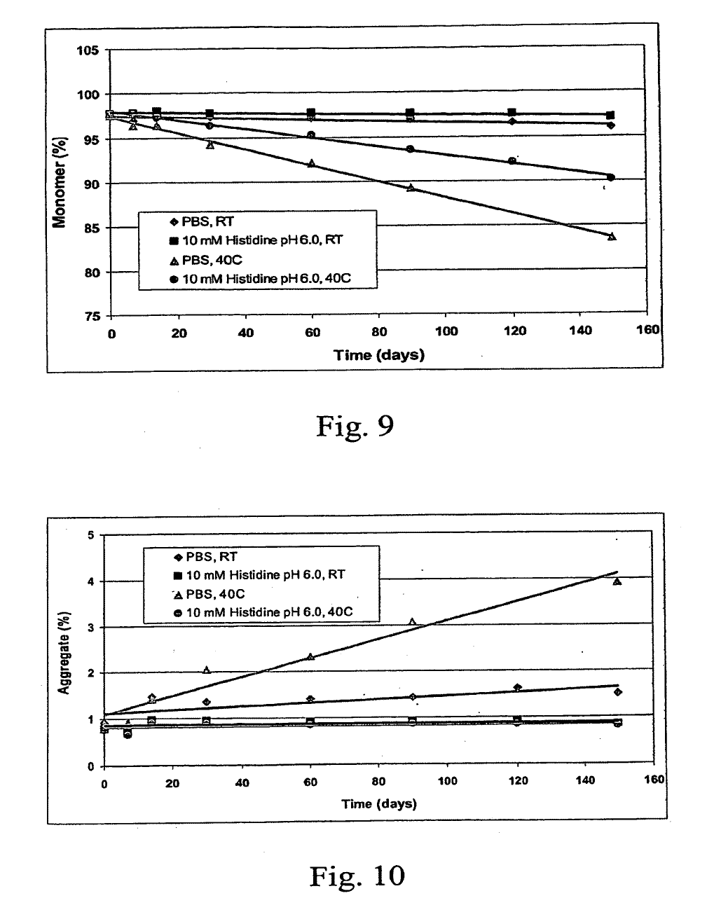

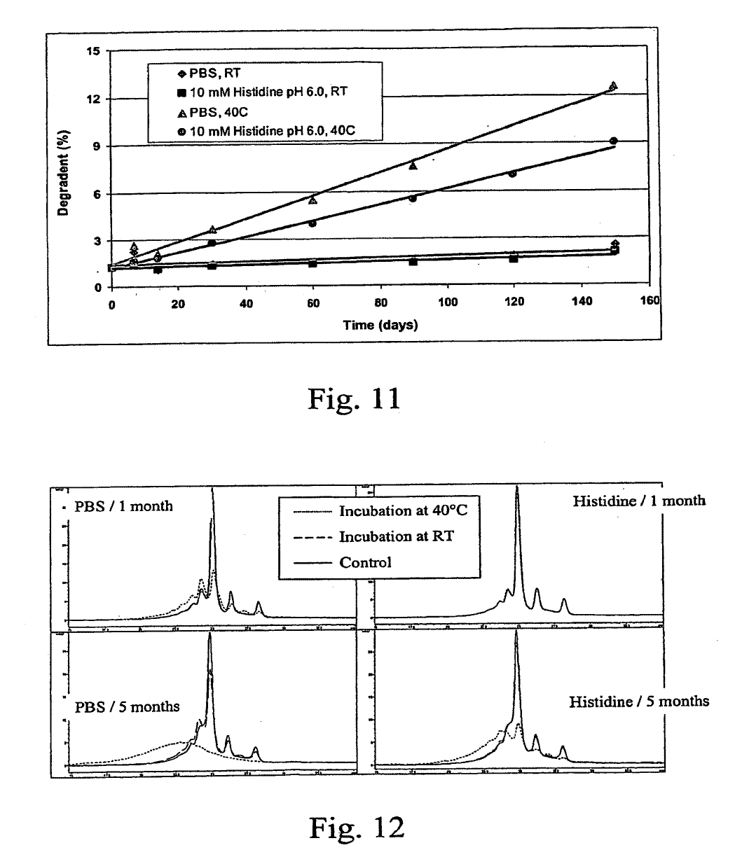

SEC-HPLC analysis: The SEC-HPLC analysis of IMC-1121B in PBS or 10 mM histidine buffer (pH 6.0) following 150 days of incubation at 40° C. and room temperature was performed according to procedure described above. The HPLC chromatograms are shown in FIG. 8. The total percent of aggregate in control, RT and 40° C. samples was 0.90, 1.49 and 3.90 for PBS and 0.80, 0.82 and 0.75 for 10 mM histidine buffer (pH 6.0), respectively. The total percent degradent in control, RT and 40° C. samples was 1.32, 2.56 and 12.54 respectively, for PBS and 1.23, 2.09 and 9.00 for 10 mM histidine buffer pH 6.0 formulations, respectively. The changes in percent monomer, percent aggregate, and percent degradent as a function of incubation time are shown in FIGS. 9, 10 and 11, respectively. Percent monomer decreased and percent aggregate and percent degradent increased at faster rate in PBS formulation than 10 mM histidine (pH 6.0). The 10 mM histidine buffer (pH 6.0) provides a superior environment for maintenance of the IMC-1121B antibody as

IEC-HPLC analysis: Ion exchange chromatography of IMC-1121B following 30 and 150 days of incubation at 40° C. and room temperature was performed on an Agilent 1100 Series LC using a Dionex ProPac WCX-10 analytical column. The samples were eluted with a linear gradient from 10 mM phopshate (pH 7.0), 20 mM NaCl to 10 mM Phosphate (pH 7.0), 100 mM NaCl in 32 minutes. The IEC-HPLC chromatograms are shown in FIG. 12. Incubation at room temperature and 40° C., caused the peaks to shift toward lower retention time (i.e. toward acidic pH) in both formulations. However, the shifts were considerably larger in the PBS formulation than in 10 mM histidine buffer (pH 6.0) formulation.

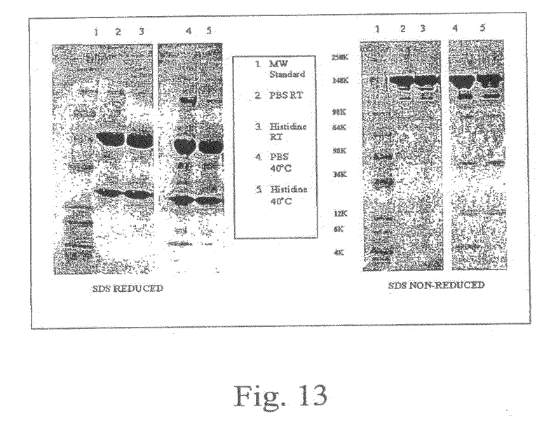

SDS-PAGE analysis: The IMC-1121B antibody (at 5 mg/mL) in PBS or 10 mM histidine buffer (pH 6.0) was incubated at room temperature or 40° C. for 150 days prior to analysis by reducing and non-reducing SDS-PAGE (4-20% tris-glycine gradient gel) according to standard protocols. The samples incubated in PBS had greater amounts of degradation products that the samples incubated in 10 mM histidine (pH 6.0) as measured by the intensity of the bands (FIG. 13).

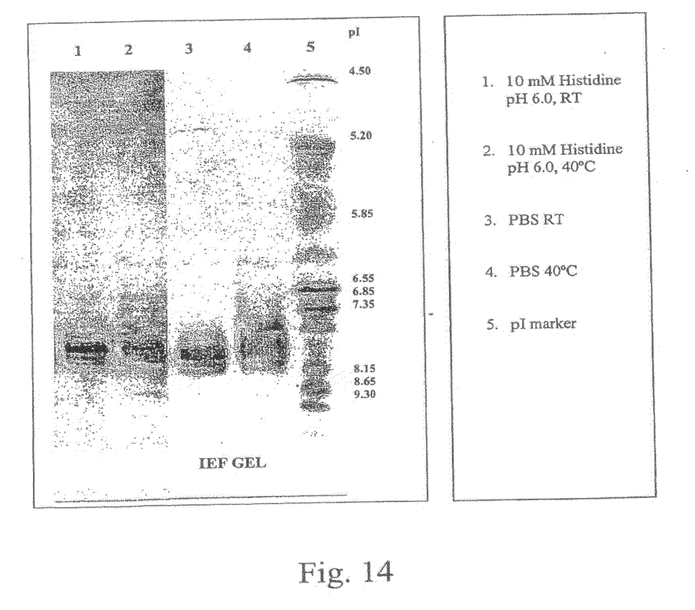

Isoelectic Focusing (IEF) analysis: IMC-1121B at 5 mg/mL in PBS and 10 mM histidine (pH 6.0) formulations after 150 days of incubation at RT and 40° C. was analyzed by IEF (pH range 6.0-10.5). Isoelectic focusing analysis was performed on IsoGel® Agarose IEF plates with a pH range from 6.0 to 10.5. The resulting bands migrated towards acidic pH both in PBS and histidine formulations. However, the shift was greater for the PBS formulation than for the 10 mM histidine (pH 6.0) formulation (FIG. 14).

### Example 4

### Freeze-Drying Formulation Screening

In the second stage of optimization, bulking agents and cryo- and lyo-protectants were optimized at a fixed antibody concentration of 20 mg/mL in 10 mM histidine buffer (pH 6.0). The additives tested were mannitol, glycine, sucrose and trehalose as shown in the design of experiment matrix (Table 2). As controls, IMC-1121B antibody at the concentration of 5 mg/mL in solution formulations (without freeze-drying) with PBS buffer (pH 6.0) or 10 mM histidine buffer (pH 6.0) was analyzed.

TABLE 2
DOE Matrix for Freeze-dried Formulation Screening

| IMC-1121B | Sucrose | Teahouse | Glycine | Mannitol |
|---|---|---|---|---|
| (mg/mL) | (%) | (%) | (%) | (%) |
| 20 | 4 | 0 | 0 | 0 |
| 20 | 0 | 4 | 0 | 0 |
| 20 | 0 | 0 | 4 | 0 |
| 20 | 0 | 0 | 0 | 4 |
| 20 | 2 | 0 | 2 | 0 |
| 20 | 2 | 0 | 0 | 2 |
| 20 | 0 | 2 | 2 | 0 |
| 20 | 0 | 20 | 0 | 2 |

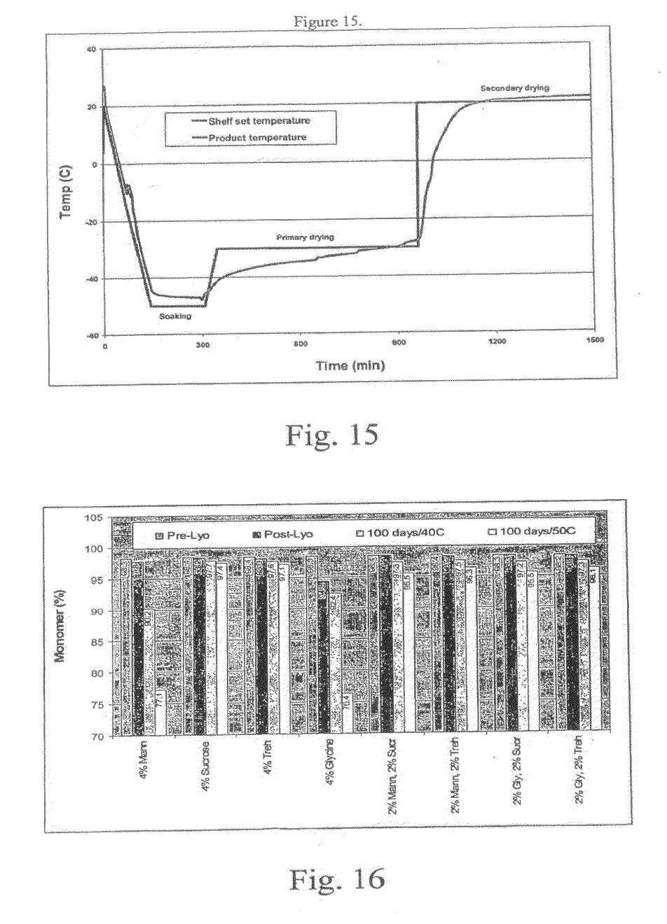

Freeze-drying Process: The products were lyophilized using a Lyostar II freeze-dryer. The lyophilzaion tray was loaded with sample at room temperature. Products were soaked at −50° C. for 2 hours. Primary drying was performed at −30° C. for 10 hours followed by secondary drying at 20° C. for another 10 hours. The cooling and heating rates were 0.5° C./min. Chamber pressure during primary and secondary drying was 50 mT. Once lyophilization was completed, the sample chamber was backfilled with N₂ and capped. The lyophilization process was completed in about 24 hours. The shelf set temperature and products temperature as a function of run time is shown in FIG. 15. The lyophilization process was considered completed when product temperature reached (or crossed) the shelf set temperature.

Accelerated temperature stability: The lyophilized antibody formulations were incubated for 100 days either at 40° C. or 50° C. After the incubation period, products were reconstituted to 5 mg/mL with 10 mM histidine buffer (pH 6.0). The reconstitution time was less than 1 min. The percent monomers remained after incubation is shown in FIG. 16. The freeze-dried formulations with 4% sucrose or 4% trehalose retained the highest percentage of monomer after the 100 day incubations at 40° C. and 50° C.

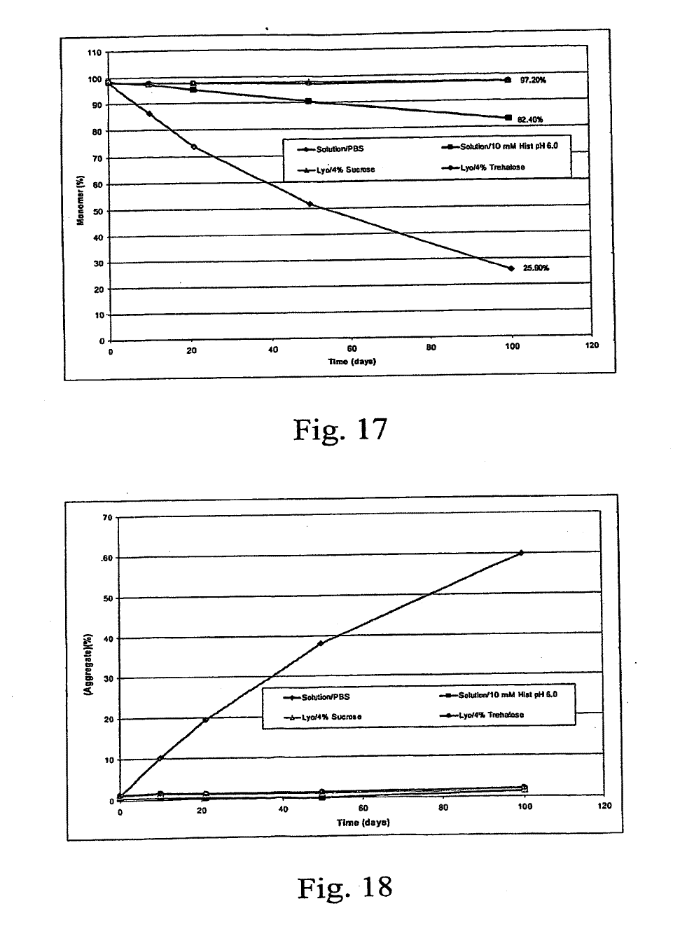

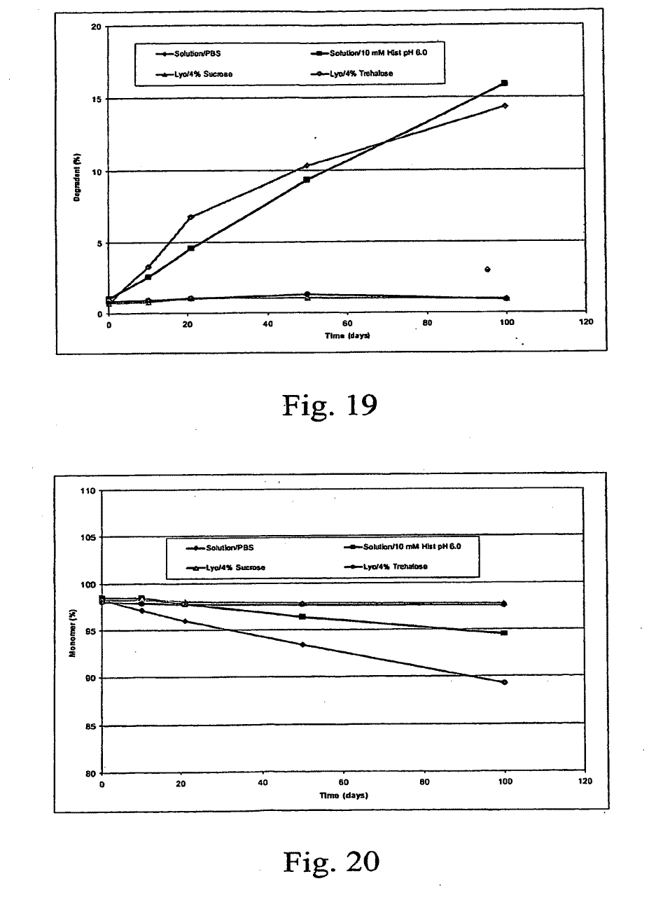

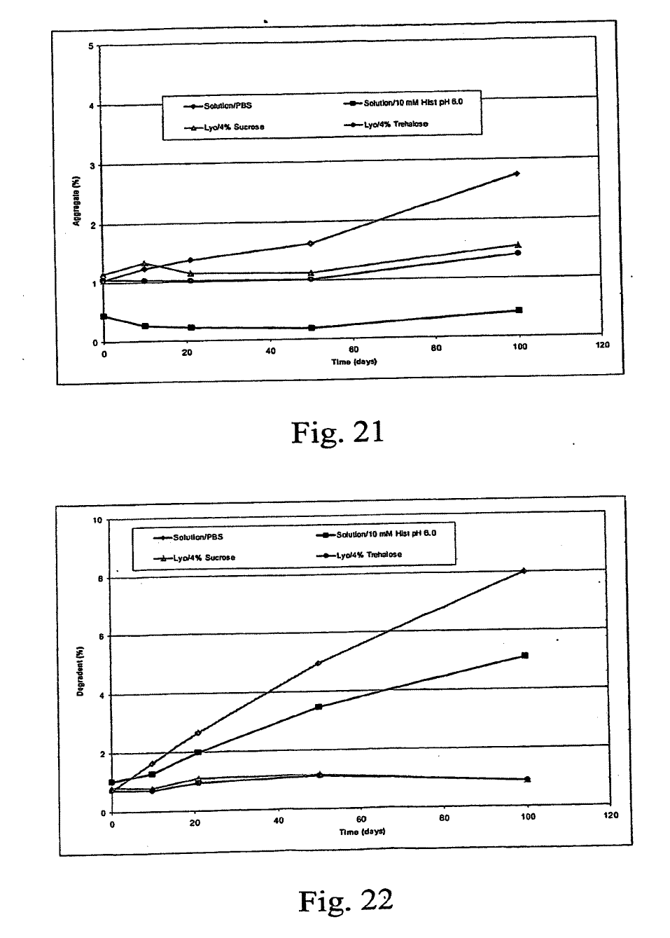

Accelerated temperature stability comparison between freeze-dried and solution formulations: The freeze-dried formulations: (1) 20 mg/mL IMC-1121B, 4% sucrose, 10 mM histidine buffer (pH 6.0), and (2) 20 mg/mL IMC-1121B, 4% trehalose, 10 mM histidine buffer (pH 6.0), was compared with solution formulations (1) 5 mg/mL IMC-1121B in PBS (pH 7.2) and (2) 5 mg/mL IMC-1121B in 10 mM histidine buffer (pH 6.0). The samples were incubated at 40° C. or 50° C. for up to 100 days. After incubation period, the lyophilized products were reconstituted to 5 mg/mL with 10 mM histidine buffer (pH 6.0). The reconstituted lyophilized samples and the solution samples were analyzed by SEC-HPLC. The variation of percent monomer, aggregate and degradent as a function of incubation time at 40° C. or 50° C. is given in FIGS. 17 through 23. Percent degradation increased with time in both the solution formulations but it remained unchanged in lyophilized formulations (FIGS. 19 and 22).

### Example 5

### Freeze-Drying Formulation for High Concentration Antibody

The previous results demonstrated that of the compounds tested, 4% sucrose or 4% trehalose provides the greatest stability for freeze-dried formulations of the IMC-1121B antibody at concentrations of 20 mg/mL. In this study we have raised IMC-1121B concentration from 20 mg/mL to 50 mg/mL and varied sucrose concentration from 4% to 8% with the goal of to formulating an IMC-1121B at a concentration of 50 mg/mL. As a control, IMC-1121B at 20 mg/mL in the presence of 4% sucrose was also lyophilized. The lyophilized products and control solution formulation were incubated at room temperature, 40° C. and 50° C. for up to 3 months. The control solution formulation consisted of the optimized, current recommended solution formulation for the IMC-1121B antibody (5 mg/mL in 10 mM histidine, 133 mM Glycine, 75 mM NaCl, 0.01% Tween 80). Following the incubation period, lyophilized products were reconstituted to 5 mg/mL with 10 mM histidine buffer (pH 6.0) and then analyzed by SEC-HPLC, IEC-HPLC, and reducing and non-reducing SDS-PAGE.

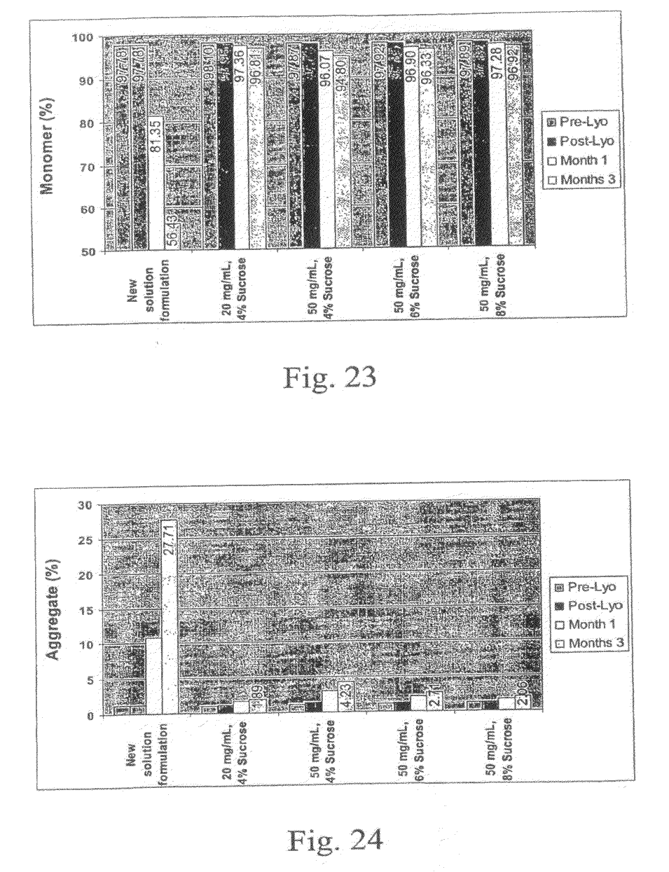

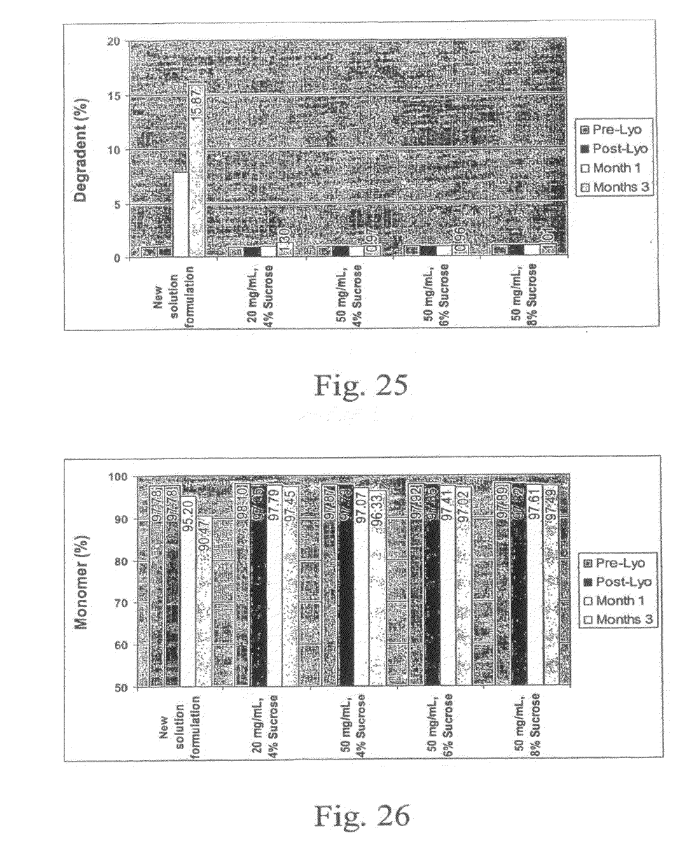

SEC-HPLC analysis of lyophilized and solution formulated IMC-1121B after 50° C. incubation: SEC-HPLC was performed on samples before and after lyophilization and following one month and 3 month incubations at 50° C. Following the incubation, the lyophilized products were reconstituted with 10 mM histidine (pH 6.0). Variation in the percent monomer, aggregate and degradent is shown in FIGS. 23, 24 and 25, respectively. The percent monomer was largest and aggregate was smallest for 8% sucrose sample. Lyophilized samples contained significantly less degradents than the solution formulated samples.

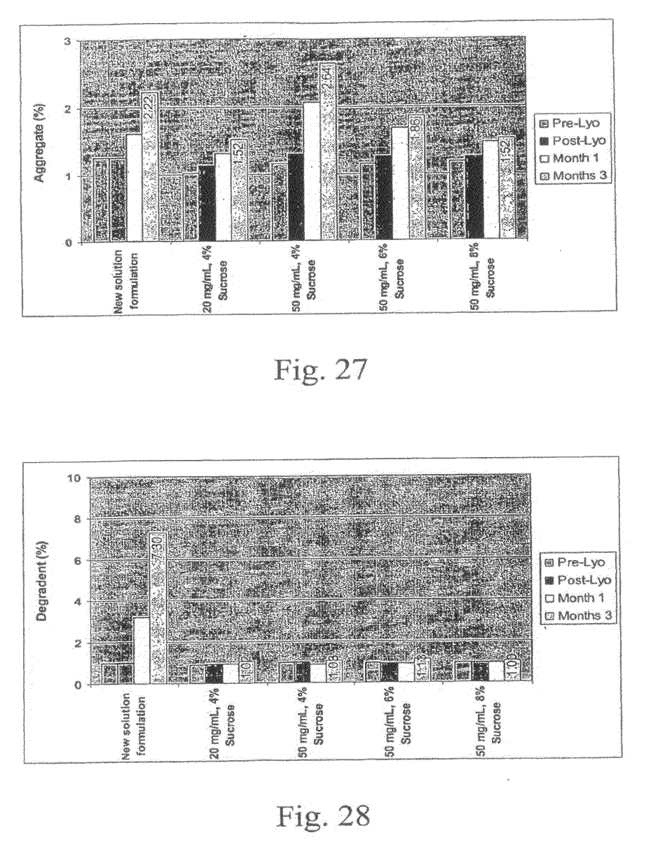

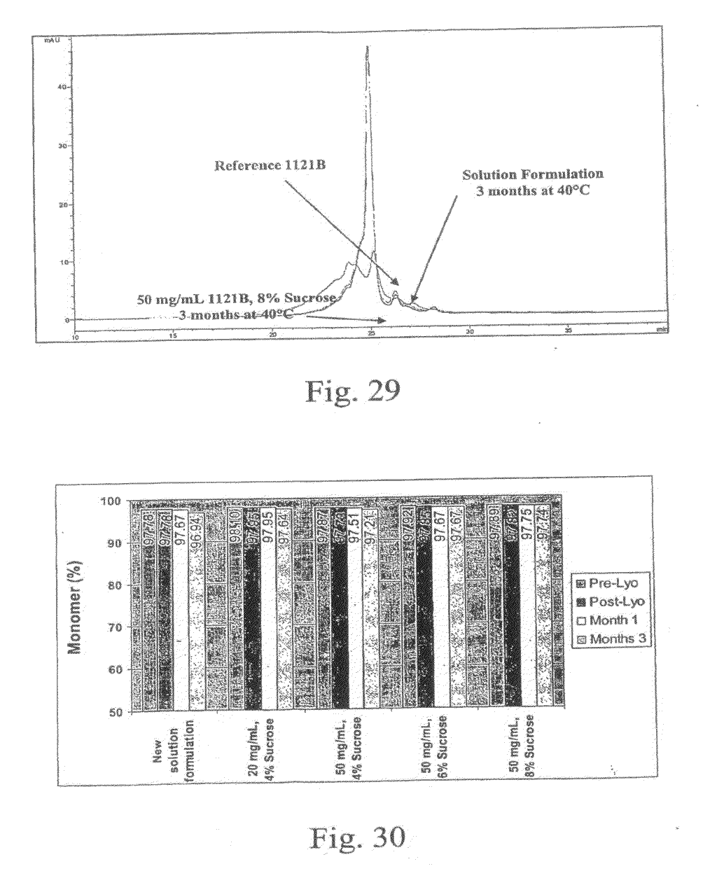

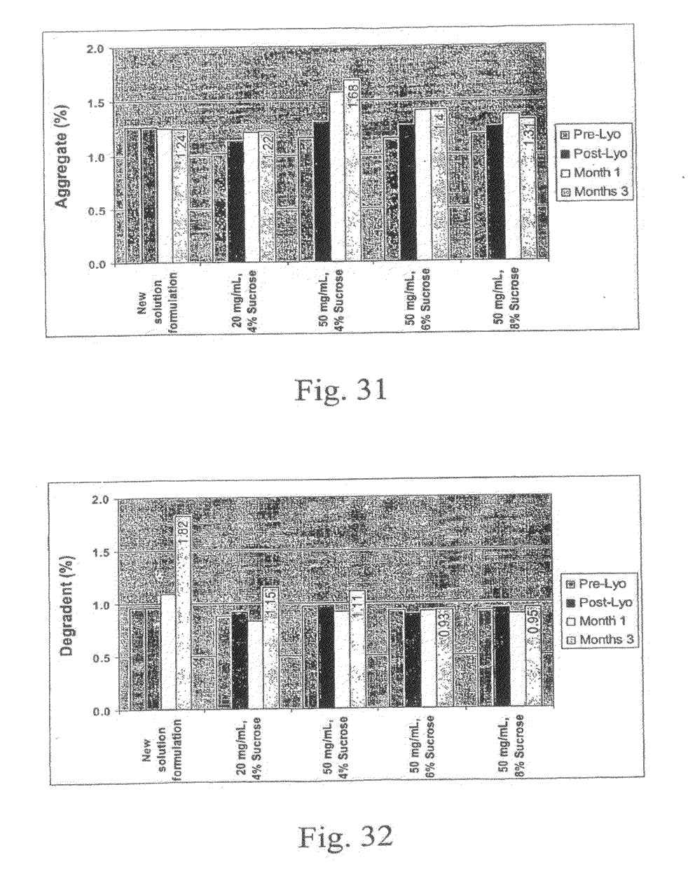

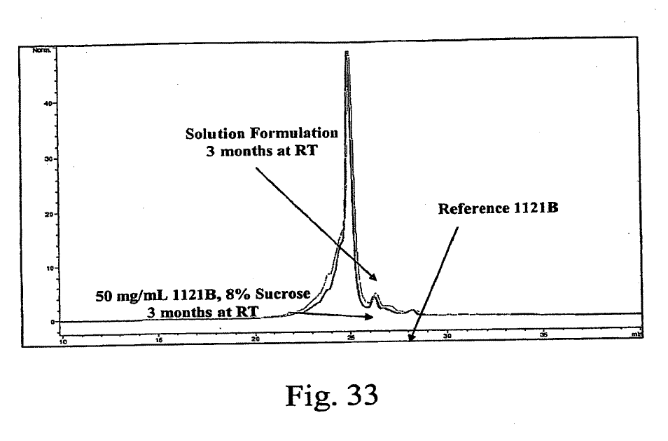

SEC-HPLC and IEC-HPLC analysis of lyophilized and solution formulated IMC-1121B after incubation at Room Temperature and at 40° C.: SEC-HPLC and IEC-HPLC were performed on samples before and after lyophilization and following one month and 3 month incubations at room temperature and 40° C. Following the incubation, the lyophilized products were reconstituted with 10 mM histidine buffer (pH 6.0). Variation of percent monomer, aggregate and degradent is shown in FIGS. 26, 27 and 28, respectively, for samples incubated at 40° C., and in FIGS. 30, 31 and 32, respectively, for samples incubated at room temperature. Lyophilized samples contained significantly less degradents than the solution formulated samples. An IEC-HPLC chromatogram of IMC-1121B incubated 3 months in solution, or freeze-dried containing 8% sucrose are shown in FIG. 29 (40° C. incubations) and FIG. 33 (room temperature incubations). A reference IMC-1121B sample was included for comparison. The chromatogram of the freeze-dried sample is similar to the reference IMC-1121B, but the chromatogram for solution formulated IMC-1121B was shifted toward acidic pH.

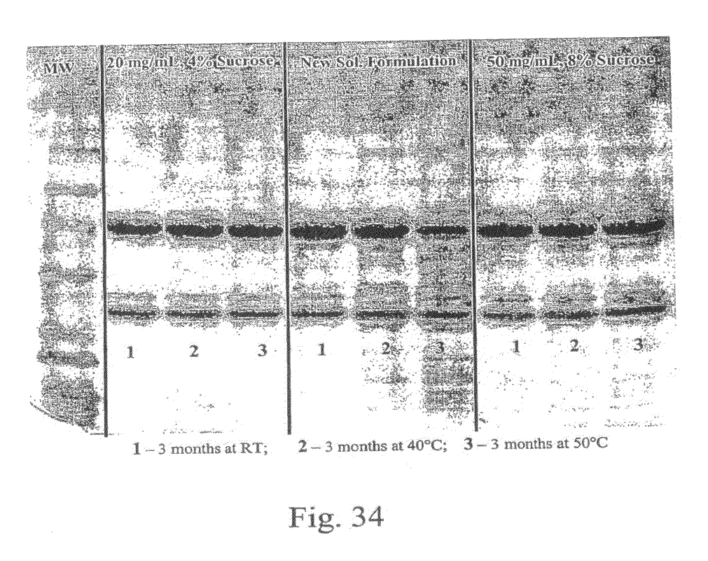

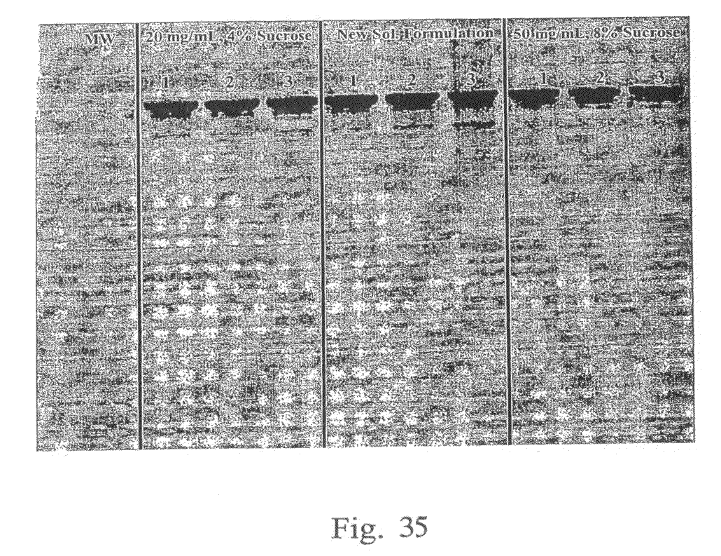

SDS-PAGE analysis of lyophilized and solution formulated IMC-1121B after a 3 months incubation: The lyophilized products were reconstituted into 10 mM histidine buffer (pH 6.0). IMC-1121B maintained in solution, and IMC-1121B reconstituted freeze-dried samples in 10 mM histidine buffer (pH 6.0) were analyzed with a 4-20% reducing SDS-PAGE (FIG. 34) and a 4-20% non-reducing SDS-PAGE (FIG. 35) following a three month incubation. The lyophilized formulations, 20 mg/ml antibody with 4% sucrose and 50 mg/ml antibody with 8% sucrose, displayed significantly reduced heavy chain degradation in comparison with the non-lyophilized formulation.

What is claimed is:

1. A stable formulation comprising an antibody and a buffer wherein non-enzymatic fragmentation of the antibody substantially reduced.

2. The formulation of claim 1, wherein the antibody is an anti-VEGFR antibody.

3. The formulation of claim 1, wherein the antibody is an anti-VEGFR2 antibody.

4. The formulation of claim 3, wherein the VEGFR2 antibody is IMC-1121B.

5. The formulation of claim 1, wherein the antibody concentration is about 50 to about 200 mg/ml.

6. The formulation of claim 1, wherein the buffer comprises a histidine buffer.

7. The formulation of claim 6, wherein the histidine buffer concentration is about 5 mM to about 50 mM.

8. The formulation of claim 6, wherein the histidine buffer concentration is about 10 mM.

9. The formulation of claim 6, wherein the histidine buffer pH is about 5.5 to about 6.5.

10. The formulation of claim 6, wherein the histidine buffer pH is about 6.0.

11. The formulation of claim 1, wherein the buffer comprises a citrate buffer.

12. The formulation of claim 11, wherein the citrate buffer pH is about 5.5 to about 6.5.

13. The formulation of claim 11, wherein the citrate buffer pH is about 6.0.

14. The formulation of claim 1, wherein the buffer comprises an acetate buffer.

15. The formulation of claim 14, wherein the acetate buffer pH is about 5.5 to about 6.5.

16. The formulation of claim 14, wherein the acetate buffer pH is about 6.0.

17. A stable lyophilized formulation comprising:
an antibody
a buffer, and
a lyoprotectant
wherein non-enzymatic fragmentation of the antibody substantially reduced.

18. The formulation of claim 17, wherein the antibody is an anti-VEGFR antibody.

19. The formulation of claim 17, wherein the antibody is an anti-VEGFR2 antibody.

20. The formulation of claim 19, wherein the VEGFR2 antibody is IMC-1121B.

21. The formulation of claim 17, wherein the antibody concentration is about 50 to about 200 mg/ml.

22. The formulation of claim 17, wherein the buffer comprises a histidine buffer.

23. The formulation of claim 22, wherein the histidine buffer concentration is about 5 mM to about 50 mM.

24. The formulation of claim 22, wherein the histidine buffer concentration is about 10 mM.

25. The formulation of claim 22, wherein the histidine buffer pH is about 5.5 to about 6.5.

26. The formulation of claim 22, wherein the histidine buffer pH is about 6.0.

27. The formulation of claim 17, wherein the buffer comprises a citrate buffer.

28. The formulation of claim 27, wherein the citrate buffer pH is about 5.5 to about 6.5.

29. The formulation of claim 27, wherein the citrate buffer pH is about 6.0.

30. The formulation of claim 17, wherein the buffer comprises an acetate buffer.

31. The formulation of claim 30, wherein the acetate buffer pH is about 5.5 to about 6.5.

32. The formulation of claim 30, wherein the acetate buffer pH is about 6.0.

33. The formulation of claim 17, wherein the lyoprotectant is a sugar.

34. The formulation of claim 33, wherein the lyoprotectant is sucrose.

35. The formulation of claim 33, wherein the lyoprotectant is trehalose.

36. The formulation of claim 17, which further comprises a surfactant.

37. The formulation of claim 36, wherein the surfactant is tween 80.

38. The formulation of claim 17, which further comprises a stabilizing agent.

39. The formulation of claim 38, wherein the stabilizing agent is aspartic acid.

40. A lyophilized formulation comprising:
an anti-VEGFR antibody,
a buffer, and
a lyoprotectant.

41. The formulation of claim 40, wherein the antibody is a VEGFR2 antibody.

42. The formulation of claim 41, wherein the VEGFR2 antibody is IMC-1121B.

43. The formulation of claim 42, wherein the antibody concentration is about 5 to about 50 mg/ml.

44. The formulation of claim 40, wherein the buffer comprises a histidine buffer.

45. The formulation of claim 44, wherein the histidine buffer concentration is about 5 mM to about 50 mM.

46. The formulation of claim 44, wherein the histidine buffer concentration is about 10 mM.

47. The formulation of claim 44, wherein the histidine buffer pH is about 5.5 to about 6.5.

48. The formulation of claim 44, wherein the histidine buffer pH is about 6.0.

49. The formulation of claim 40, wherein the buffer comprises a citrate buffer.

50. The formulation of claim 40, wherein the buffer comprises an acetate buffer.

51. The formulation of claim 40, wherein the lyoprotectant is a sugar.

52. The formulation of claim 51, wherein the lyoprotectant is sucrose.

53. The formulation of claim 51, wherein the lyoprotectant is trehalose.

54. The formulation of claim 40, which further comprises a surfactant.

55. The formulation of claim 54, wherein the surfactant is tween 80.

56. The formulation of claim 40, which further comprises a stabilizing agent.

57. The formulation of claim 56, wherein the stabilizing agent is aspartic acid.

58. A lyophilized formulation comprising:
an anti-VEGFR2 antibody,
a histidine buffer, and
a lyoprotecting sugar.

59. The formulation of claim 58, wherein the VEGFR2 antibody is IMC-1121B.

60. The formulation of claim 58, wherein the histidine buffer pH is about 5.5 to about 6.5.

61. The formulation of claim 58, wherein the histidine buffer pH is about 6.0.

62. The formulation of claim 58, wherein the lyoprotectant is sucrose.

63. The formulation of claim 58, wherein the lyoprotectant is trehalose.

64. The formulation of claim 58, which further comprises a surfactant.

65. The formulation of claim 64, wherein the surfactant is tween 80.

66. The formulation of claim 58, which further comprises a stabilizing agent.

67. The formulation of claim 66, wherein the stabilizing agent is aspartic acid.

68. A, method of treatment comprising administering a reconstituted formulation comprising:
an anti-VEGFR antibody,
a buffer,
and a lyoprotectant.


SEQUENCE LISTING (5 sequences)

SEQ ID NO: 1 - DNA, 321 bp, Homo sapiens
  CDS (1)..(321)
gaaattgtgatgacacagtctccagccaccctgtctttgtctccaggggaaagagccaccctctcctgcagggccagtcagagtgttagcagctacttagcctggtaccaacagaaacctggccaggctcccaggctcctcatctatgattcatccaacagggccactggcatcccagccagattcagtggcagtgggtctgggacagacttcactctcaccatcagcagcctagagcctgaagattttgcaacttattactgtctacagcataacacttttcctccgacgttcggccaagggaccaaggtggaaatcaaa

SEQ ID NO: 2 - protein, 107 aa, Homo sapiens
EIVMTQSPATLSLSPGERATLSCRASQSVSSYLAWYQQKPGQAPRLLIYDSSNRATGIPARFSGSGSGTDFTLTISSLEPEDFATYYCLQHNTFPPTFGQGTKVEIK

SEQ ID NO: 3 - DNA, 348 bp, Homo sapiens
  CDS (1)..(321)
gaggtgcagctggtgcagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagtcacagatgcttttgatatctggggccaagggacaatggtcaccgtctcaagc

SEQ ID NO: 4 - protein, 107 aa, Homo sapiens
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWG

SEQ ID NO: 5 - protein, 465 aa, Homo sapiens
MGWSCIILFLVATATGVHSEVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK